(19) United States
(12) Patent Application Publication
(10) Pub. No.: US 2024/0329181 A1
(43) Pub. Date: Oct. 3, 2024

(54) AUDIO TARGETING FOR OPTIMAL LOCATION OF MICROPHONE TO RECORD BIOMETRIC SIGNALS FOR TELEMEDICINE

(71) Applicant: Optum, Inc., Minnetonka, MN (US)

(72) Inventors: Russell Henry Amundson, Johns Island, SC (US); Andrey Volozin, Redmond, WA (US)

(21) Appl. No.: 18/461,132

(22) Filed: Sep. 5, 2023

Related U.S. Application Data
(60) Provisional application No. 63/492,583, filed on Mar. 28, 2023.

(51) Int. Cl. G01S 3/802; A61B 5/00; A61B 7/00

(52) U.S. Cl. CPC G01S 3/802; A61B 5/7455; A61B 7/003

(57) ABSTRACT

Embodiments provide for confirming whether biometric recording data structures that are obtained at a same target listening site relative to a patient body as measurements previously obtained by a medical professional or other first or previous listener. Accordingly, embodiments herein ensure that detected changes in biometric recording data structures are a result of changes in anatomic conditions and not due to variations in listening sites.

### CROSS-REFERENCE TO RELATED APPLICATIONS

The present application claims priority to U.S. Provisional Application Ser. No. 63/492,583, titled “AUDIO TARGETING FOR OPTIMAL LOCATION OF MICROPHONE TO RECORD LUNG OR HEART SOUNDS FOR TELEMEDICINE,” filed Mar. 28, 2023, the contents of which are incorporated herein by reference in their entirety.

### BACKGROUND

Telemedicine requires patient data to be acquired from a location remote from the consulting provider. One such form of data is an audio file which provides information about organ or anatomic component (e.g., heart, lung, artery, or the like) sounds, typically obtained from an electronic stethoscope or other listening device (e.g., a microphone on a mobile cell phone or computing device). The location on the body where the listening device is placed can influence the quality and audio characteristics of the lung or heart sound. A non-clinical listening device user such as a patient or family member may not have real time feedback in order to place the listening device at the optimal location. The optimal location is where a professional listening device user such as a physician has obtained the optimal audio file for lung or heart sounds.

Through applied effort, ingenuity, and innovation, many of these identified deficiencies and problems have been solved by developing solutions that are structured in accordance with embodiments of the present disclosure, many examples of which are described in detail herein.

### BRIEF SUMMARY

Embodiments provide for confirming whether biometric recording data structures that are obtained at a same target listening site relative to a patient body as measurements previously obtained by a medical professional or other first or previous listener. Accordingly, embodiments herein ensure that detected changes in biometric recording data structures are a result of changes in anatomic conditions and not due to variations in listening sites.

In various embodiments, a system comprises memory and one or more processors communicatively coupled to the memory, the one or more processors configured to receive a first biometric recording data structure associated with a first patient listening site associated with a patient body, receive a second biometric recording data structure associated with a second patient listening site associated with the patient body, generate a first listening vector based on the first biometric recording data structure and a second listening vector based on the second biometric recording data structure, and responsive to determining, based on the first listening vector and the second listening vector, that the second patient listening site is different from the first patient listening site, initiate one or more guidance-related actions at a recording device to direct the recording device toward the first patient listening site for obtaining a subsequent biometric recording data structure.

In various embodiments, one or more non-transitory computer-readable storage media are provided, including instructions that, when executed by one or more processors, cause the one or more processors to receive a first biometric recording data structure associated with a first patient listening site, receive a second biometric recording data structure associated with a second patient listening site, generate a first listening vector based on the first biometric recording data structure and a second listening vector based on the second biometric recording data structure, and responsive to determining, based on the first listening vector and the second listening vector, that the second patient listening site is different from the first patient listening site, initiate one or more guidance-related actions at a recording device to direct the recording device toward the first patient listening site.

In various embodiments, a computer-implemented method comprises receiving, by one or more processors, a first biometric recording data structure associated with a first patient listening site, receiving, by the one or more processors, a second biometric recording data structure associated with a second patient listening site, generating, by the one or more processors, a first listening vector based on the first biometric recording data structure and a second listening vector based on the second biometric recording data structure, and responsive to determining, based on the first listening vector and the second listening vector, that the second patient listening site is different from the first patient listening site, initiating, by the one or more processors, one or more guidance-related actions at a recording device to direct the recording device toward the first patient listening site. In some embodiments, the first listening vector may include both audio and visual data (e.g., based on data obtained using audio capture devices and video capture devices such as a camera).

The above summary is provided merely for purposes of summarizing some example embodiments to provide a basic understanding of some aspects of the present disclosure. Accordingly, it will be appreciated that the above-described embodiments are merely examples and should not be construed to narrow the scope or the spirit of the present disclosure in any way. It will be appreciated that the scope of the present disclosure encompasses many potential embodiments in addition to those here summarized, some of which will be further described below. Other features, aspects, and advantages of the subject matter will become apparent from the description, the drawings, and the claims.

### BRIEF DESCRIPTION OF THE DRAWINGS

Having thus described certain example embodiments of the present disclosure in general terms above, non-limiting and non-exhaustive embodiments of the subject disclosure will now be described with reference to the accompanying drawings which are not necessarily drawn to scale. The components illustrated in the accompanying drawings may or may not be present in certain embodiments described herein. Some embodiments may include fewer (or more) components than those shown in the drawings. Some embodiments may include the components arranged in a different way:

FIG. 1 provides an example overview of a system that can be used to practice embodiments of the present disclosure.

FIG. 2 provides an example device guidance computing entity in accordance with some embodiments discussed herein.

FIG. 3 provides an example external computing entity in accordance with some embodiments discussed herein.

FIGS. 4A, 4B, 4C, 4D, 4E, 4F, 4G, 4H, and 4I depict an example of placement of a listening device and one or more constant sound sources, in accordance with embodiments of the present disclosure.

FIGS. 5A and 5B depict example operations for performing in accordance with embodiments herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not necessarily indicate being based only on or based solely on the referenced element or elements unless so indicated. Like numbers refer to like elements throughout. Moreover, while certain embodiments of the present disclosure are described with reference to device guidance, one of ordinary skill in the art will recognize that the disclosed concepts can be used to perform other types of data analysis.

### I. Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware framework and/or operating system platform. A software component comprising assembly language instructions may require conversion into executable machine code by an assembler prior to execution by the hardware framework and/or platform. Another example programming language may be a higher-level programming language that may be portable across multiple frameworks. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as in a particular directory, folder, or library. Software components may be static (e.g., pre-established or fixed) or dynamic (e.g., created or modified at the time of execution).

A computer program product may include non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid-state drive (SSD)), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatuses, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments can produce specifically configured machines performing the steps or operations specified in the block diagrams and flowchart illustrations. Accordingly, the block diagrams and flowchart illustrations support various combinations of embodiments for performing the specified instructions, operations, or steps.

### II. Example Framework

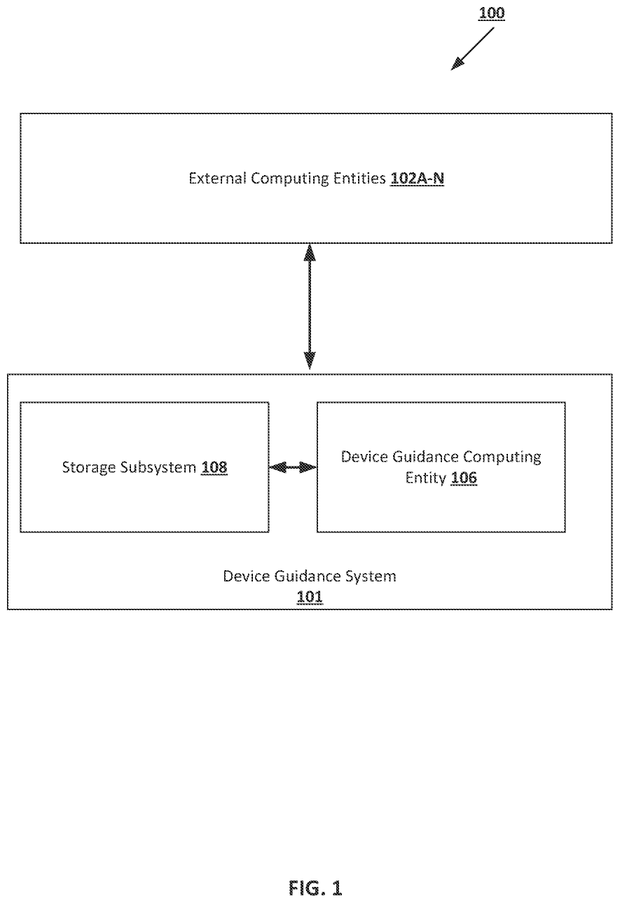

FIG. 1 provides an example overview of an architecture that can be used to practice embodiments of the present disclosure. The architecture 100 may include a device guidance system 101 and a device guidance computing entity 106 configured to generate outputs that can be used to perform one or more output-based actions. The device guidance system 101 may communicate with one or more external computing entities 102A-N using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (e.g., network routers, and/or the like).

The system 100 includes a storage subsystem 108 configured to store at least a portion of the data utilized by the device guidance system 101. The device guidance computing entity 106 may be in communication with the external computing entities 102A-N. The device guidance computing entity 106 may be configured to: (i) train one or more machine learning models based on a training data store stored in the storage subsystem 108, (ii) store trained machine learning models as part of a model definition data store of the storage subsystem 108, (iii) utilize trained machine learning models to perform an action, and/or the like.

In one example, the device guidance computing entity 106 may be configured to generate a prediction (e.g., with respect to patient condition), classification, and/or any other data insight based on data provided by an external computing entity such as external computing entity 102A, external computing entity 102B, and/or the like. It will be appreciated that an external computing entity 102A may include a listening device in order to obtain biometric recordings and provide the same to the device guidance computing entity 106.

The storage subsystem 108 may be configured to store the model definition data store and the training data store for one or more machine learning models. The device guidance computing entity 106 may be configured to receive requests and/or data from at least one of the external computing entities 102A-N, process the requests and/or data to generate outputs (e.g., predictive outputs, classification outputs, and/or the like), and provide the outputs to at least one of the external computing entities 102A-N. In some embodiments, the external computing entity 102A, for example, may periodically update/provide raw and/or processed input data to the device guidance system 101. The external computing entities 102A-N may further generate user interface data (e.g., one or more data objects) corresponding to the outputs and may provide (e.g., transmit, send, and/or the like) the user interface data corresponding with the outputs for presentation to the external computing entity 102A (e.g., to an end-user).

The storage subsystem 108 may be configured to store at least a portion of the data utilized by the device guidance computing entity 106 to perform one or more steps/operations and/or tasks described herein. The storage subsystem 108 may be configured to store at least a portion of operational data and/or operational configuration data including operational instructions and parameters utilized by the device guidance computing entity 106 to perform the one or more steps/operations described herein. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

The device guidance computing entity 106 can include an analysis engine and/or a training engine. The analysis engine may be configured to perform one or more data analysis techniques. The training engine may be configured to train the analysis engine in accordance with training data stored in the storage subsystem 108.

### Example Device Guidance Computing Entity

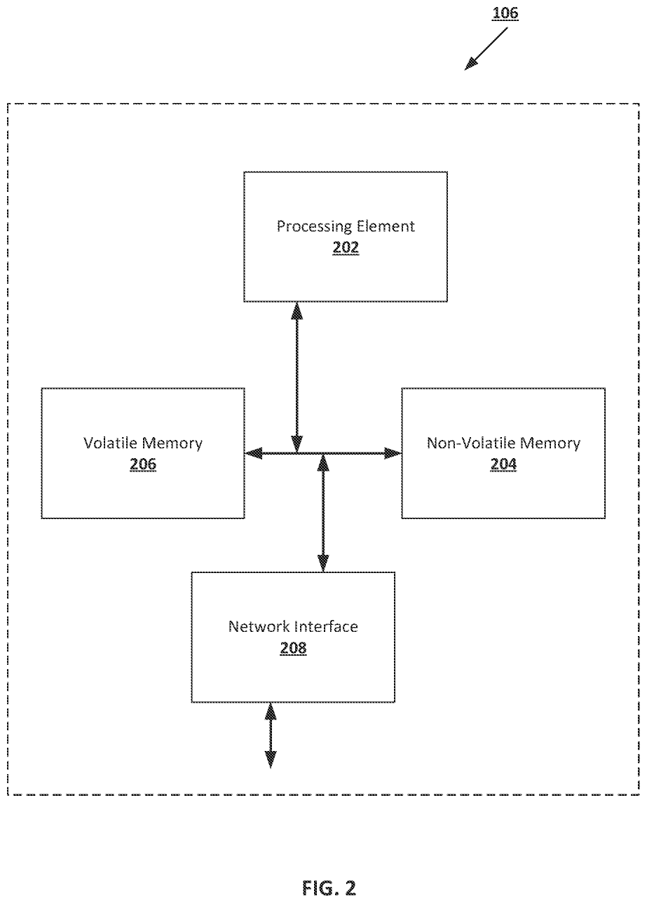

FIG. 2 provides an example device guidance computing entity 106 in accordance with some embodiments discussed herein. In general, the terms computing entity, computer, entity, device, system, and/or similar words used herein interchangeably may refer to, for example, one or more computers, computing entities, desktops, mobile phones, tablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, steps/operations, and/or processes described herein. Such functions, steps/operations, and/or processes may include, for example, transmitting, receiving, operating on, processing, displaying, storing, determining, creating/generating, monitoring, evaluating, comparing, and/or similar terms used herein interchangeably. In one embodiment, these functions, steps/operations, and/or processes can be performed on data, content, information, and/or similar terms used herein interchangeably.

The device guidance computing entity 106 may include a network interface 208 for communicating with various computing entities, such as by communicating data, content, information, and/or similar terms used herein interchangeably that can be transmitted, received, operated on, processed, displayed, stored, and/or the like.

In one embodiment, the device guidance computing entity 106 may include or be in communication with a processing element 202 (also referred to as processors, processing circuitry, and/or similar terms used herein interchangeably) that communicate with other elements within the device guidance computing entity 106 via a bus, for example. As will be understood, the processing element 202 may be embodied in a number of different ways including, for example, as at least one processor/processing apparatus, one or more processors/processing apparatuses, and/or the like.

For example, the processing element 202 may be embodied as one or more complex programmable logic devices (CPLDs), microprocessors, multi-core processors, coprocessing entities, application-specific instruction-set processors (ASIPs), microcontrollers, and/or controllers. Further, the processing element 202 may be embodied as one or more other processing devices or circuitry. The term circuitry may refer to an entirely hardware embodiment or a combination of hardware and computer program products. Thus, the processing element 202 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 202 may be configured for a particular use or configured to execute instructions stored in one or more memory elements including, for example, one or more volatile memories 206 and/or non-volatile memories 204. As such, whether configured by hardware or computer program products, or by a combination thereof, the processing element 202 may be capable of performing steps or operations according to embodiments of the present disclosure when configured accordingly. The processing element 202, for example in combination with the one or more volatile memories 206 and/or or non-volatile memories 204, may be capable of implementing one or more computer-implemented methods described herein. In some implementations, the device guidance computing entity 106 can include a computing apparatus, the processing element 202 can include at least one processor of the computing apparatus, and the one or more volatile memories 206 and/or non-volatile memories 204 can include at least one memory including program code. The at least one memory and the program code can be configured to, upon execution by the at least one processor, cause the computing apparatus to perform one or more steps/operations described herein.

The non-volatile memories 204 (also referred to as non-volatile storage, memory, memory storage, memory circuitry, media, and/or similar terms used herein interchangeably) may include at least one non-volatile memory device 204, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile memories 204 may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

The one or more volatile memories (also referred to as volatile storage, memory, memory storage, memory circuitry, media, and/or similar terms used herein interchangeably) can include at least one volatile memory 206 device, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile memories 206 may be used to store at least portions of the databases, database instances, database management systems, data, applications, programs, program modules, scripts, source code, object code, byte code, compiled code, interpreted code, machine code, executable instructions, and/or the like being executed by, for example, the processing element 202. Thus, the databases, database instances, database management systems, data, applications, programs, program modules, scripts, source code, object code, byte code, compiled code, interpreted code, machine code, executable instructions, and/or the like may be used to control certain embodiments of the operation of the device guidance computing entity 106 with the assistance of the processing element 202.

As indicated, in one embodiment, the device guidance computing entity 106 may also include the network interface 208 for communicating with various computing entities, such as by communicating data, content, information, and/or the like that can be transmitted, received, operated on, processed, displayed, stored, and/or the like. Such communication data may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the device guidance computing entity 106 may be configured to communicate via wireless client communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

### Example External Computing Entity

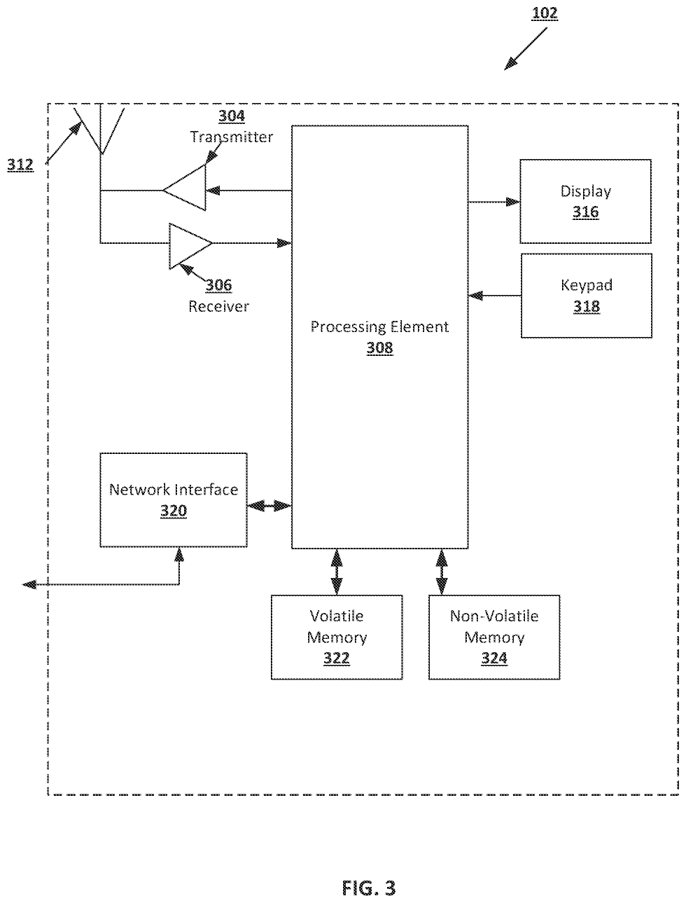

FIG. 3 provides an example external computing entity 102A in accordance with some embodiments discussed herein. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, steps/operations, and/or processes described herein. The external computing entities 102A-N can be operated by various parties. As shown in FIG. 3, the external computing entity 102A can include an antenna 312, a transmitter 304 (e.g., radio), a receiver 306 (e.g., radio), and/or an external entity processing element 308 (e.g., CPLDs, microprocessors, multi-core processors, coprocessing entities, ASIPs, microcontrollers, and/or controllers) that provides signals to and receives signals from the transmitter 304 and the receiver 306, correspondingly. As will be understood, the external entity processing element 308 may be embodied in a number of different ways including, for example, as at least one processor/processing apparatus, one or more processors/processing apparatuses, and/or the like as described herein with reference the processing element 202.

The signals provided to and received from the transmitter 304 and the receiver 306, correspondingly, may include signaling information/data in accordance with air interface standards of applicable wireless systems. In this regard, the external computing entity 102A may be capable of operating with one or more air interface standards, communication protocols, modulation types, and access types. More particularly, the external computing entity 102A may operate in accordance with any of a number of wireless communication standards and protocols, such as those described above with regard to the device guidance computing entity 106. In a particular embodiment, the external computing entity 102A may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the external computing entity 102A may operate in accordance with multiple wired communication standards and protocols, such as those described above with regard to the device guidance computing entity 106 via an external entity network interface 320.

Via these communication standards and protocols, the external computing entity 102A can communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The external computing entity 102A can also download changes, add-ons, and updates, for instance, to its firmware, software (e.g., including executable instructions, applications, program modules), operating system, and/or the like.

According to one embodiment, the external computing entity 102A may include location determining embodiments, devices, modules, functionalities, and/or the like. For example, the external computing entity 102A may include outdoor positioning embodiments, such as a location module adapted to acquire, for example, latitude, longitude, altitude, geocode, course, direction, heading, speed, universal time (UTC), date, and/or various other information/data. In one embodiment, the location module can acquire data such as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the DecimalDegrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating a position of the external computing entity 102A in connection with a variety of other systems, including cellular towers, Wi-Fi access points, and/or the like. Similarly, the external computing entity 102A may include indoor positioning embodiments, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning embodiments can be used in a variety of settings to determine the location of someone or something to within inches or centimeters.

The external computing entity 102A may include a user interface 316 (e.g., a display, speaker, and/or the like) that can be coupled to the external entity processing element 308. In addition, or alternatively, the external computing entity 102A can include a user input interface 319 (e.g., keypad, touch screen, microphone, and/or the like) coupled to the external entity processing element 308).

For example, the user interface 316 may be a user application, browser, and/or similar words used herein interchangeably executing on and/or accessible via the external computing entity 102A to interact with and/or cause the display, announcement, and/or the like of information/data to a user. The user input interface 318 can comprise any of a number of input devices or interfaces allowing the external computing entity 102A to receive data including, as examples, a keypad (hard or soft), a touch display, voice/speech interfaces, motion interfaces, and/or any other input device. In embodiments including a keypad, the keypad can include (or cause display of) the conventional numeric (0-9) and related keys (#, *, and/or the like), and other keys used for operating the external computing entity 102A and may include a full set of alphabetic keys or set of keys that may be activated to provide a full set of alphanumeric keys. In addition to providing input, the user input interface 318 can be used, for example, to activate or deactivate certain functions, such as screen savers, sleep modes, and/or the like.

The external computing entity 102A can also include one or more external entity non-volatile memories 322 and/or one or more external entity volatile memories 324, which can be embedded within and/or may be removable from the external computing entity 102A. As will be understood, the external entity non-volatile memories 322 and/or the external entity volatile memories 324 may be embodied in a number of different ways including, for example, as described herein with reference the non-volatile memories 204 and/or the external volatile memories 206.

### III. Examples of Certain Terms

The terms “subject body” or “patient body” may refer to a user or subject of an analysis in accordance with embodiments herein. In some examples, the subject body or patient body is a human or other live subject.

The term “auscultation” refers to the act of listening to sounds arising within organs or anatomic components (such as the lungs, heart, arteries, and the like) as an aid to diagnosis and treatment.

The term “tele-auscultation” refers to providing a remote auscultation to another place. In some examples, tele-auscultation involves transmitting an audio recording obtained during an auscultation event to a location remote from the site of auscultation. In some examples, the audio recording may include a biometric recording data structure.

The term “crackling lung sound” refers to audibly detectable (e.g., using a measuring device such as a stethoscope) bubbling, clicking, or rattling sounds in the lungs. Crackling lung sounds occur when a person breathes in and the small airways open. A medical professional may hear the crackling lung sounds as someone inhales and may classify them as coarse or fine. Crackling can be associated with asthma, bronchitis, cystic fibrosis, heart disease, lung infections, pericarditis, pneumonia, pulmonary fibrosis, and more.

The terms “rhonchi lung sounds” or “rhonchi” refer to audibly detectable (e.g., using a measuring device such as a stethoscope) lung sounds that resemble snoring. Rhonchi sounds occur when something blocks the airways or when airflow becomes turbulent as it passed through the large airways. Rhonchi sounds can occur in expiration, or both inspiration and expiration, but rarely solely in inspiration.

The term “stridor lung sounds” refers to audibly detectable (e.g., using a measuring device such as a stethoscope) lung sounds that are wheeze-like. Usually, an airflow blockage in the windpipe or the back of the throat causes stridor lung sounds. Unlike wheezes, stridor occurs during inspiration and is typically only in the upper airways. Stridor can be associated with bronchitis, croup, epiglottitis, hemangioma, infection of the trachea, laryngomalacia, a narrow voice box, a paralyzed vocal cord, and more.

The term “rub lung sounds” refers to audibly detectable (e.g., using a measuring device such as a stethoscope) lung sounds that result from inflamed pleura rubbing together. Pleura are protective, cushioning layers of tissue. Pleura cover the outside of the lungs and the inside of the chest wall. If the pleura become inflamed, they can rub against each other, causing an adventitious breath sound. Rubs of the pleura are typically louder than the other sounds because they occur on the chest wall.

The term “wheeze lung sounds” or “wheezes” refer to audibly detectable (e.g., using a measuring device such as a stethoscope) lung sounds that are high-pitched. Air movement through constricted narrow airways cause wheezes. Like rhonchi, wheezes can happen as someone breathes out or breathes in or out, but rarely occurs just when someone breathes in. Wheezing can be associated with allergies, asthma, bronchitis, chronic obstructive pulmonary disorder (COPD), epiglottitis, gastroesophageal reflux (GERD), heart failure, lung cancer, obstruction of the voice box or windpipe, pneumonia, respiratory syncytial virus (RSV), sleep apnea, and more.

The terms “device,” “remote patient monitoring (RPM) device,” “measuring device,” “listening device,” “biometric listening or recording device,” or “recording device” refer to one or more devices configured to obtain physiological, anatomic, or other measurements from a patient body. In some embodiments, the physiological, anatomic or other measurements comprise audio. The recording device is one or more of a stethoscope, a mobile computing device, a remote monitoring device, an audio capture device, a video capture device, or a computing device.

In some embodiments, a listening or recording device may include one or more transducers to transduce sound emanating through the body surface into an electronic signal. This may be a smart phone with a microphone, an “electronic stethoscope” or a home listening device of low cost yet adequate capability to obtain an analyzable audio signal. The listening or recording device may further include a processing component (e.g., a processor) that can capture and retain in memory the electronic signal from the one or more transducers via a wired connection or through a wireless connectivity. The processing component may be configured to record the captured electronic signal, compare the retained or recorded signal to a previously retained or recorded signal, and/or analyze the difference between the current retained or recorded signal and the previously retained or recorded signal. The listening device may optionally include a display component that can display the difference between the current retained or recorded signal and a previous signal. In other embodiments, the processing component is configured to transmit (e.g., with communications circuitry) the captured electronic signal to a device guidance system in according with embodiments herein. The processing component may be configured to receive guidance-related actions from a device guidance system and automatically executed them (e.g., provide haptic, audio, visual feedback). In some embodiments, the listening device may include more than one device, for example an audio capture device as well as a video capture device.

The term “guidance-related action” refers to one or more of haptics, audio notification, or visual notification that are automatically provided via a listening device based on a determination that the listening device should be moved to a different listening location.

The term “biometric recording data structure” refers to one or more items of data associated with a recording obtained using a listening device associated with a patient body. In some embodiments, the biometric recording data structure is an audio recording file. In some embodiments, the audio recording file contains lung sounds associated with the patient body.

The terms “patient listening site,” “target listening site,” or “listening site” refer to a location of a patient body to which a listening device is placed. In some embodiments, the patient listening site is on the skin of a patient and in a chest region of the patient.

The term “listening vector” refers to a data structure comprising a plurality of records, each representative of a feature of a biometric recording data structure. For example, a vector of AR1,2 may be generated (e.g., in accordance with a distance from the listening device to a right-side sound emitter of the constant sound source) and a vector AL1,2 may be generated (e.g., in accordance with a distance from the listening device to a left-side sound emitter of the constant sound source). A vector of BR1,2 may be generated (e.g., in accordance with a distance from the listening device to a right-side sound emitter of the constant sound source) and a vector BL1,2 may be generated (e.g., in accordance with a distance from the listening device to a left-side sound emitter of the constant sound source). In some embodiments, the listening vector may include both audio and visual data (e.g., based on data obtained using audio capture devices and video capture devices such as a camera).

The term “constant sound source” refers to a device configured to emit a sound into or near a patient body. In some embodiments a constant sound source device may include multiple sound emitters (e.g., one on a first or right-hand side and one on a second or left-hand side of the device). The multiple sound emitters may be spaced apart by a distance (e.g., ˜2 inches) and the device may be adhered to the skin of the patient body with an adhesive pad (e.g., similar to what is used for EKG electrodes). In some embodiments, the constant sound source may include ultrasound.

The term “constant sound source site” refers to a location of a patient body to which a constant sound source is adhered to placed.

The term “feedback” refers to haptics, vibrations, audio notification, or visual notification electronically provided via a listening or recording device. The term “tactile feedback” refers to haptics and/or vibrations.

The terms “device guidance model” or “predictive models” refer to a data structure or model specifically configured or designed to identify one or more changes in anatomic conditions associated with a patient body based on features of a biometric recording data structure (and/or changes between a first biometric recording data structure and a second biometric recording data structure). In some embodiments, the one or more predictive models are trained based on historical anatomic data associated with a plurality of patient bodies.

The terms “trained machine learning model,” “machine learning model,” “model,” “one or more models,” or “ML” refer to a machine learning or deep learning task or mechanism. Machine learning is a method used to devise complex models and algorithms that lend themselves to prediction. A machine learning model is a computer-implemented algorithm that may learn from data with or without relying on rules-based programming. These models enable reliable, repeatable decisions and results and uncovering of hidden insights through machine-based learning from historical relationships and trends in the data. In some embodiments, the machine learning model is a clustering model, a regression model, a neural network, a random forest, a decision tree model, a classification model, or the like.

A machine learning model is initially fit or trained on a training dataset (e.g., a set of examples used to fit the parameters of the model). The model may be trained on the training dataset using supervised or unsupervised learning. The model is run with the training dataset and produces a result, which is then compared with a target, for each input vector in the training dataset. Based on the result of the comparison and the specific learning algorithm being used, the parameters of the model are adjusted. The model fitting may include both variable selection and parameter estimation. Successively, the fitted model is used to predict the responses for the observations in a second dataset called the validation dataset. The validation dataset provides an unbiased evaluation of a model fit on the training dataset while tuning the model's hyperparameters (e.g., the number of hidden units in a neural network). In some embodiments, the model can be trained and/or trained in real-time (e.g., online training) while in use.

The machine learning models as described herein may make use of multiple ML engines, e.g., for analysis, transformation, and other needs. The system may train different ML models for different needs and different ML-based engines. The system may generate new models (based on the gathered training data) and may evaluate their performance against the existing models. Training data may include any of the gathered information, as well as information on actions performed based on the various recommendations.

The ML models may be any suitable model for the task or activity implemented by each ML-based engine. Machine learning models may be some form of neural network. The underlying ML models may be learning models (supervised or unsupervised). As examples, such algorithms may be prediction (e.g., linear regression) algorithms, classification (e.g., decision trees, k-nearest neighbors) algorithms, time-series forecasting (e.g., regression-based) algorithms, association algorithms, clustering algorithms (e.g., K-means clustering, Gaussian mixture models, DBscan), or Bayesian methods (e.g., Naïve Bayes, Bayesian model averaging, Bayesian adaptive trials), image to image models (e.g., FCN, PSPNet, U-Net) sequence to sequence models (e.g., RNNs, LSTMs, BERT, Autoencoders) or Generative models (e.g., GANs).

Alternatively, ML models may implement statistical algorithms, such as dimensionality reduction, hypothesis testing, one-way analysis of variance (ANOVA) testing, principal component analysis, conjoint analysis, neural networks, support vector machines, decision trees (including random forest methods), ensemble methods, and other techniques. Other ML models may be generative models (such as Generative Adversarial Networks or auto-encoders).

In various embodiments, the ML models may undergo a training or learning phase before they are released into a production or runtime phase or may begin operation with models from existing systems or models. During a training or learning phase, the ML models may be tuned to focus on specific variables, to reduce error margins, or to otherwise optimize their performance. The ML models may initially receive input from a wide variety of data, such as the gathered data described herein.

The terms “data,” “content,” “digital content,” “digital content object,” “signal,” “information,” and similar terms may be used interchangeably to refer to data capable of being transmitted, received, and/or stored in accordance with embodiments of the present disclosure. Thus, use of any such terms should not be taken to limit the spirit and scope of embodiments of the present disclosure. Further, where a computing device is described herein to receive data from another computing device, it will be appreciated that the data may be received directly from another computing device or may be received indirectly via one or more intermediary computing devices, such as, for example, one or more servers, relays, routers, network access points, base stations, hosts, and/or the like, sometimes referred to herein as a “network.” Similarly, where a computing device is described herein to send data to another computing device, it will be appreciated that the data may be transmitted directly to another computing device or may be transmitted indirectly via one or more intermediary computing devices, such as, for example, one or more servers, relays, routers, network access points, base stations, hosts, and/or the like.

### IV. Overview, Technical Improvements, and Technical Advantages

Embodiments herein provide for breath sound analysis algorithms that pave the way to tele-auscultation capability with a remote patient monitoring (RPM) device (e.g., stethoscope), including the use of location matching over sound matching to avoid improper diagnoses due to sound quality issues. Embodiments herein may be deployed to initiate interventions for enterprise advocacy teams to reduce pneumonia (e.g., and other conditions) recurrence and reduce readmissions.

Approximately 15% of patients discharged from the hospital after treatment for community acquired pneumonia (CAP) will develop recurrent pneumonia requiring re-admission. Significant costs and morbidity are associated with recurrence. Recurrent pneumonia is associated with retained mucous in the airways of the lung. This condition can be detected by analyzing lung and breath sounds and locating a characteristic sound referred to rhonchi-a resonant, rattling sound.

Telemedicine may provide for remote patient monitoring to assess changes in health parameters. These changes can provide the basis for prediction of disease progression and recognition of disease progression could provide for early intervention to prevent decompensation. Decompensation can result in hospital re-admission, increased outpatient cost of care, member disability or demise. Monitoring lung sounds is a unique form of remote patient monitoring and abnormal lung sounds may correlate with altered lung anatomy. The altered lung anatomy may not immediately result in altered lung function—for example a pleural rub may be detected but pulmonary function may not decline until the pleural pain inhibits breathing rate or depth or severely constricts lung expansion.

Tele-auscultation (e.g., using cell phones or stethoscopes) is an effective means of detecting rhonchi. Coupled with prompt intervention after rhonchi detection, recurrent pneumonia can be averted by instituting pulmonary therapy. Existing tele-auscultation systems rely on manual guidance from a remote professional and/or sound matching. That is, a medical professional may manually listen, via a remote computing device, to audible lung sounds recorded by a remote user (e.g., a patient or someone assisting the patient). The medical professional may rely on her or his training and experience to determine an optimal listening site for recording the lung sounds and verbally instruct the remote user to move the recording device to a different location. Relying on a listener at a far end of a remote transmission line may introduce uncertainty based on the quality of the remote transmission line, noise or other errors introduced on either end of the transmission line, and differences in experience associated with the listening medical professional. Remote listening may also be impacted by loss of sound quality due to transmission distance and other factors. This uncertainty may lead to overlooked problematic health conditions and may result in the delayed provision of necessary medical care.

A home-based listening device may detect a change in lung sounds distinct from baseline lung sounds detected by a professional in a clinic setting or prior to hospital discharge. The challenge with such techniques is confirming if the detected altered lung sound indicates a change in lung function or a change in the listening site. Embodiments herein overcome such challenges and more by programmatically confirming that the listening site is a constant (e.g., consistent enough) between the new site/home use and the baseline site/professional site use, so that alteration in lung sound can be correctly attributed to alteration in lung function. Embodiments herein further ensure the listening site remains consistent or constant by providing real-time feedback (e.g., via sound or haptics) to a user obtaining the subsequent recording to direct their placement of the listening device over the correct listening site.

### V. Example System Operations

Embodiments herein provide for the acquisition and recording of an audio file from a patient from a specific body location. Acoustic wave form characteristics of the audio file are analyzed or processed and then saved, tagging them to the patient and the specific body location. The specific body location is referred to herein as the target listening site for subsequent recordings or measurements. Subsequent recordings from the patient are also analyzed, where the subsequent recordings are obtained by the patient (e.g., or other user) and remote from a physician location (e.g., hospital or doctor's office). Using localization techniques described herein, notifications may be provided to a device used by the patient to assist with optimal obtaining of the subsequent recordings. The notifications provide guidance as to whether the proposed listening location is within a threshold distance of the target listening site. If the listening device is not within a threshold distance of the target listening site, the localizing techniques herein provide electronic feedback to guide the listening device in a direction to bring it closer to the target listening site. In some example embodiments, the original audio file is obtained as a baseline by a professional medical provider, which is compared to a recording or measurement obtained by a non-professional or home user or the patient. In embodiments, the biometric recording or listening device may obtain sounds emitted from a given internal organ or anatomic component of the patient (e.g., the lungs, heart, arteries, and the like).

Embodiments herein further utilize an external constant sound source to avoid any variations that may arise from the use of a different body part (e.g., the heart) as a sound source or in any variations that may arise in the transmission of sound of the different body part (e.g., heart) through a lung altered by progressive disease, which could result in increased fluid content in the lung tissues and therefore effect a change in sound transmission quality.

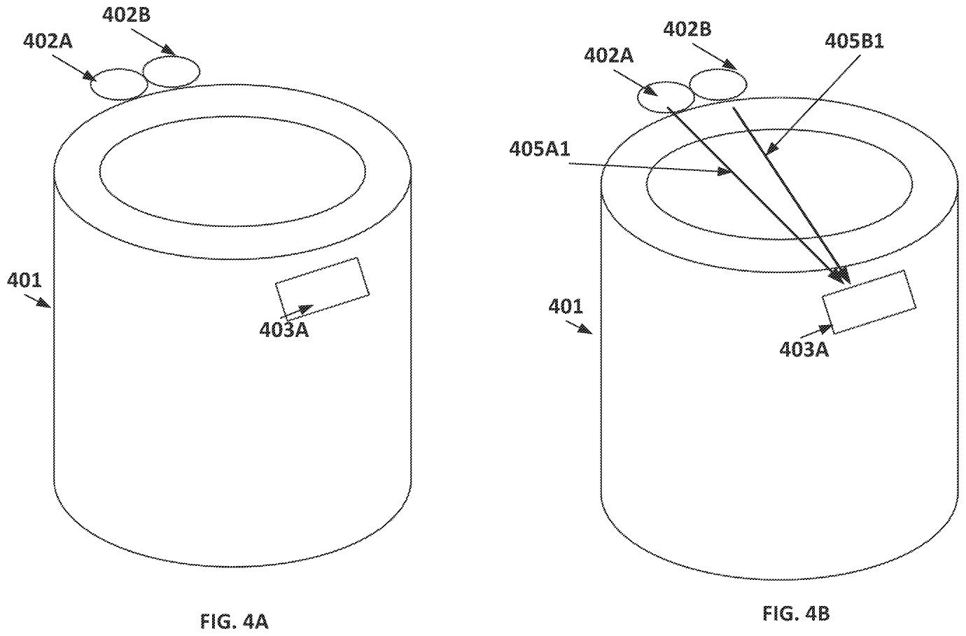

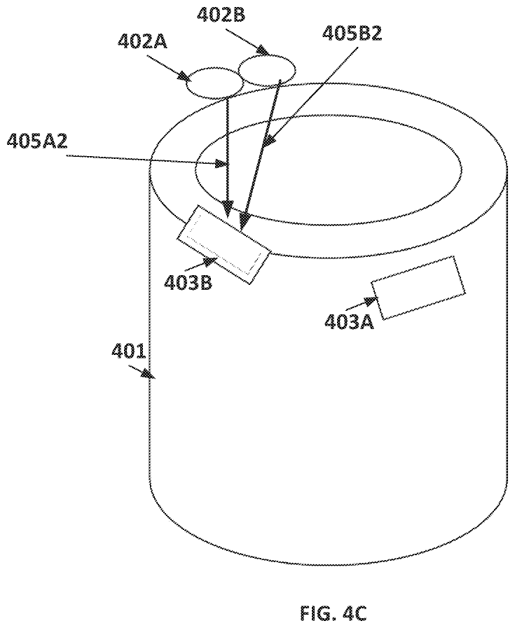

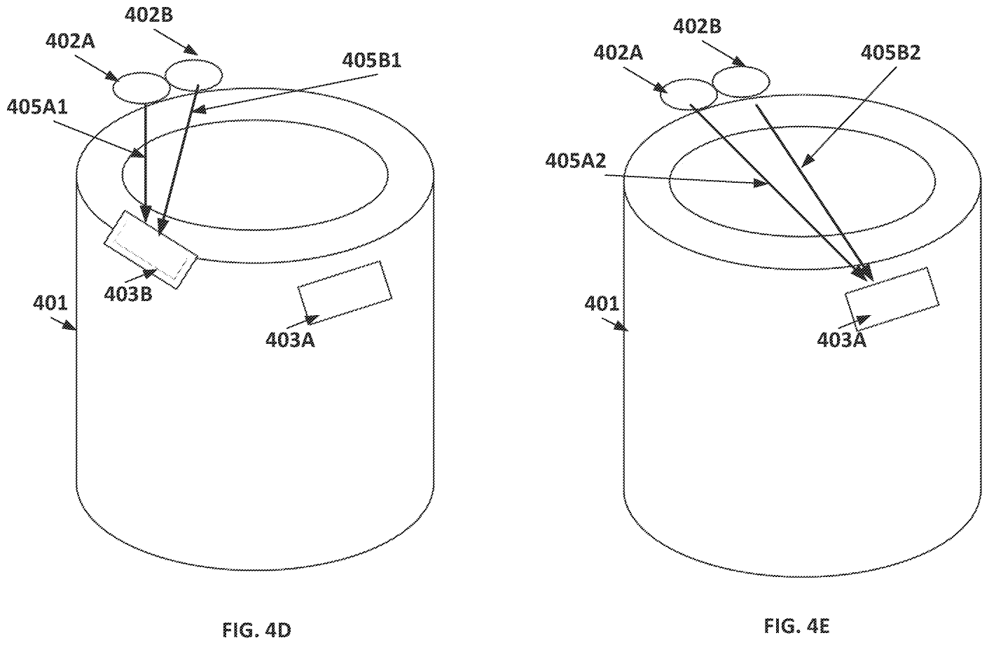

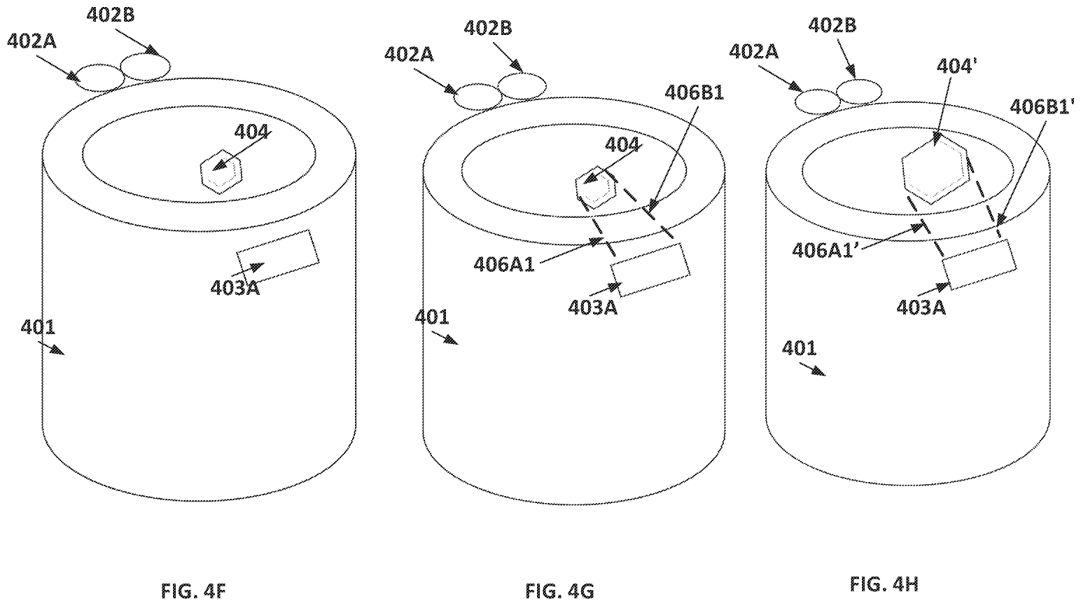

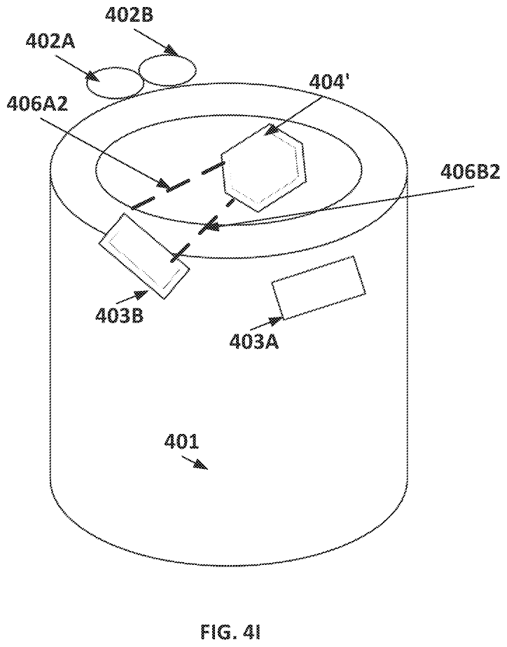

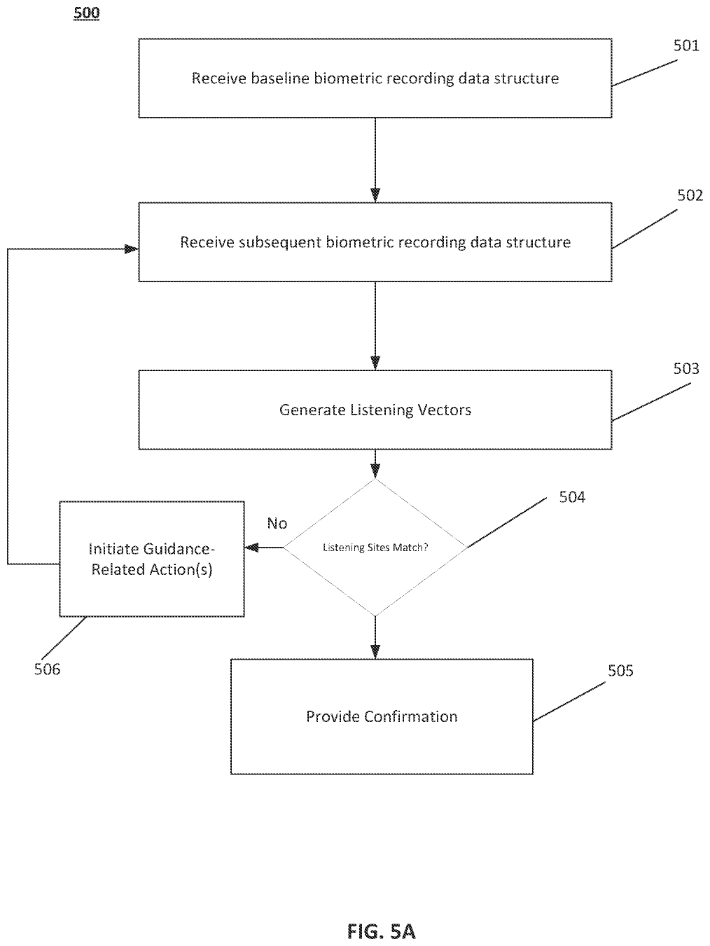

FIGS. 4A-4I depict example placement of a listening device and one or more constant sound sources, in accordance with embodiments of the present disclosure. One or more constant sound source devices 402A, 402B are used in conjunction with a biometric listening or recording device 403A (e.g., a biometric listening or recording device at a first location associated with 403A). The one or more constant sound source devices 402A, 402B are placed externally on a particular location of a patient body 401 (e.g., the “sternal notch” the indentation just above the breastbone on the front of the chest). In the case of multiple constant sound source devices, they may be placed close together or relatively far apart from one another. In some embodiments, each constant sound source device 402A, 402B may include multiple sound emitters (e.g., one on a first or right-hand side and one on a second or left-hand side of the device). The multiple sound emitters may be spaced apart by a distance (e.g., ˜2 inches) and the device may be adhered to the skin of the patient body with an adhesive pad (e.g., similar to what is used for EKG electrodes).

In some embodiments, the biometric listening or recording device 403A is a stethoscope, a mobile computing device (e.g., a mobile phone), or other computing device. In some embodiments, the biometric listening or recording device 403A may include an array of transducers (e.g., microphones or audio capture devices). The array of transducers may be configured according to a pattern (e.g., for example a pattern resembling a compass with a transducer in the “NORTH” position, a transducer in the “SOUTH” position, a transducer in the “WEST” position, and a transducer in the “EAST” position). In some example embodiments, the biometric listening or recording device measures about four inches in diameter (e.g., a size easily held in one hand).

In some embodiments, the listening device 403A comprises a single transducer. An original audio file may be converted herein into two directional vectors, one to each of the two sound sources of the sound source device 402A or 402B. On subsequent listening events, the single transducer listening device 403A can once again calculate the two directional vectors. Vector analysis can determine the differences between the two vector sets and determine the need for listening device movement, both extent and direction based on the vector set analysis.

Referring to FIG. 4B, in some embodiments, the listening device 403A is placed at a location relative to patient body 401, and a first directional vector 405A1 may be associated with a signal from constant sound source 402A (between constant sound source 402A and listening device 403A), while a second directional vector 405B1 may be associated with a signal from constant sound source 402B (between constant sound source 402B and listening device 403A).

Referring to FIG. 4C, it will be appreciated that, if the listening device 403A is instead placed at location 403B (e.g., listening device at a different location relative to body 401), vector 405A2 will differ from vector 405A1 and vector 405B2 will differ from vector 405B1. FIGS. 4D and 4E depict this comparison.

Continuing with reference to FIG. 4F, the listening device 403A is placed near or in contact with the patient body 401, in order to obtain recordings or audio associated with a sound source 404 within the body (e.g., the lung or other physical condition). In such a configuration, it can be seen that the listening device 403A is separated by distance/vectors 406A1 and 406B1 from the sound source 404 in the body.

In FIG. 4F, at a time subsequent the measurements obtained in the configuration depicted in FIGS. 4A-E, the constant sound sources 402A, 402B are preferably placed at the same location relative to the patient body 401. The listening device 403A is also placed near or in contact with the patient body 401, in order to obtain subsequent recordings or audio associated with sound source 404. Based on data stored in accordance with the measurements of FIGS. 4A-E, it can be determined whether vectors 406A1 and 406B1 are associated with the same listening location. If not, electronic guidance can be provided to the listening device 403A in a subsequent recording posture to eliminate the possibility that detected changes in lung sounds are a result of a difference in position of the listening device as opposed to changes in anatomic conditions.

In FIG. 4H, it can be seen that the clinical condition has worsened such that characteristics of the sound source within the body 404′ (different from or larger than 404) have changed. Accordingly, vectors 406A1′ and 406B1′ likely differ from 406A1 and 406B1. Further, had listening device 403B been at a different location, vectors 406A2 and 406B2 would differ from 406A1 and 406B1.

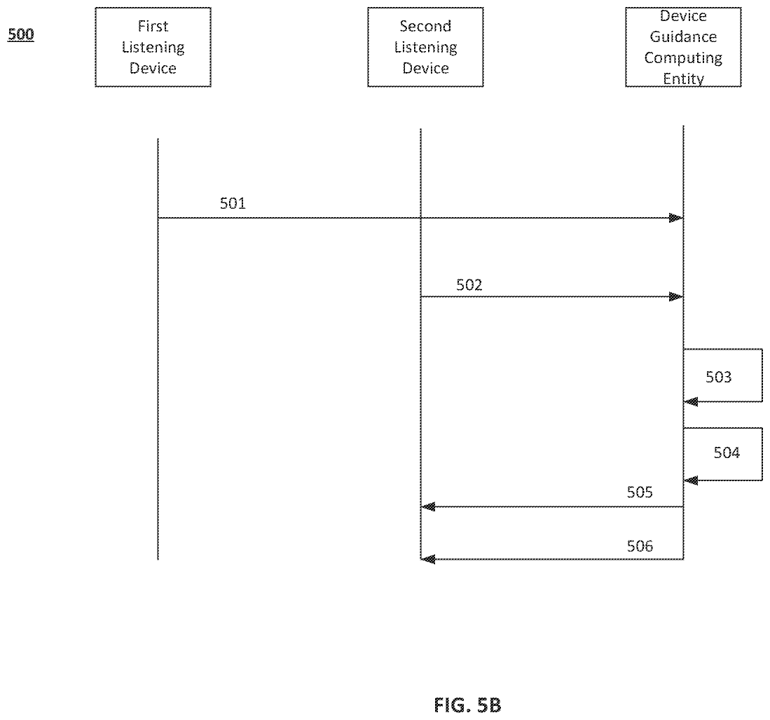

FIGS. 5A and 5B depict example operations for performing in accordance with embodiments herein. Via the various steps/operations of the process 500, the device guidance computing entity 106 can efficiently and effectively ensure that a listening device obtaining subsequent recordings associated with a patient organ or anatomic component (e.g., the lungs, heart, arteries, or the like) is in a target listening location, and thereby ensure that variations in the subsequent recordings are a result in a change in physical condition of the patient and/or patient organ. In doing so, embodiments herein provide for early detection of changes in conditions while avoiding incorrect analysis due to incorrect listening device placement.

The process 500 begins at step/operation 501 when the device guidance computing entity 106 receives a baseline biometric recording file. In some embodiments, the baseline biometric recording file is acquired at a first listening site of a patient body (e.g., “Listening Site A”). In such an example, at a constant sound source location, a constant sound source is activated to emit a sound from a first sound emitter (e.g., a right side) and then a second sound emitter (e.g., a left side). In embodiments, an audio signal obtained at a first listening site (e.g., “Listening Site A”), where a biometric listening or recording device is placed, can include an amplitude and frequency spectrum. The audio signal may be analyzed, in accordance with techniques herein, to establish a distance from each of one or more transducers (e.g., one or more microphones) of the biometric listening or recording device placed at the first listening site (e.g., “Listening Site A”) to the constant sound source location. In some examples, a vector of AR1,2 is generated (e.g., in accordance with a distance from the listening device to a right-side sound emitter of the constant sound source) and a vector AL1,2 is generated (e.g., in accordance with a distance from the listening device to a left-side sound emitter of the constant sound source).

The process 500 continues at step/operation 502, when the device guidance computing entity 106 receives a subsequent biometric recording file. In embodiments, one or more subsequent recordings may be obtained in a separate setting (e.g., a non-clinical or home setting, obtained by a different user, professional, or non-professional). In such subsequent recordings, a constant sound source is placed at (e.g., or within a threshold distance of) the constant sound source location on the patient body. A biometric listening or recording device is then placed at a second listening site (e.g., “Listening Site B”). The constant sound source is activated to emit a sound from a first sound emitter (e.g., a right-side sound emitter) and a second sound emitter (e.g., a left-side sound emitter).

The process 500 continues at step/operation 503, when the device guidance computing entity 106 analyzes the subsequent recording to generate listening vectors; that is, establish a distance from each of one or more transducers (e.g., one or more microphones) of the biometric listening or recording device placed at the second listening site (e.g., “Listening Site B”) to the constant sound source location. In some examples, a vector of BR1,2 is generated (e.g., in accordance with a distance from the listening device to a right-side sound emitter of the constant sound source) and a vector BL1,2 is generated (e.g., in accordance with a distance from the listening device to a left-side sound emitter of the constant sound source).

The process 500 continues at step/operation 504, when the device guidance computing entity 106 determines whether the subsequent recording is obtained from the first listening site. For example, the device guidance computing entity 106 determines whether the first vector associated with the right-side emitter (e.g., AR1,2) is equivalent to (e.g., or within a threshold measure of) the second vector associated with the right-side emitter (e.g., BR1,2), and whether the second vector associated with the left-side emitter (e.g., AL1,2) is equivalent to (e.g., or withing a threshold measure of) the second vector associated with the left-side emitter (e.g., BL1,2). In some embodiments, each of the features of each vector is compared against a corresponding feature of the corresponding subsequent vector (e.g., AR1 v. BR1; AR2 v. BR2; AL1 v. BL1; AL2 v. BL2). If one or more of the vectors is not a match (e.g., or within an acceptable threshold distance), the difference is treated herein as an indication that the listening device location needs to be adjusted.

The process continues at step/operation 505, in the event that the device guidance computing entity 106 determines that the vectors are equivalent or within a threshold measure of one another, then the second listening site is considered to be the same as (e.g., or acceptably within a threshold distance of) the first listening site. In some embodiments, once the vectors associated with the measurements recorded in accordance with the first listening site and the vector associated with the measurements recorded in accordance with the second listening site are equivalent or within a margin or threshold distance from one another (e.g., AR1,2=BR1,2 AND AL1,2=BL1,2), an indicator provides confirmation (e.g., via visual, audio, or haptic feedback) that the two listening sites are the same. Further analysis can compare the audio files for any difference with confidence that any detected differences are due to alteration in the lung sounds themselves and not due to alteration in the listening site during comparison. To be clear, subsequently, biometric recordings (e.g., lung sounds) can be acquired and it can be confirmed that any changes in sounds are attributed to changes in physical condition as opposed to changes in listening sites. Accordingly, any variation in sounds obtained at the second listening site as compared to sounds obtained at the first listening site may be attributed to changes in organ (e.g., lung) condition as opposed to changes in listening site.

Alternatively, the process 500 may continue at step/operation 506, in the event that the device guidance computing entity 106 determines that the vectors are not equivalent or within a threshold measure of one another, meaning the second listening site is not the same as (e.g., or acceptably within a threshold distance of) the first listening site. At step/operation 506, the device guidance computing entity 106 may cause automatic provision of guidance-related actions at the listening device. In some embodiments, guidance-related actions may include automated haptics (e.g., vibrations), automated audible sounds (e.g., chirping according to a particular tone or pattern), automated visual cues, or the like. In some embodiments, a photo image indicating the position of the listening device relative to body landmarks is acquired and stored. During the subsequent listening session, a live video capture indicates the location of the listening device. Comparing the current location to the recorded or target location indicates any deviation and the user is visually notified of the needed correction.

In some embodiments, in addition to an indication of the listening device location needing to be adjusted to match the original listening location, the direction and extent of necessary movement to have the vectors appropriately match can be generated. That is, in some examples an indicator on the back of the listening device which faces away from the skin (e.g., such as an LED, with an adjustable size arrow) can provide both direction and extent of movement guidance.

Confirmation of the second listening site being equivalent (e.g., or within a threshold distance of) the first listening site provides for confidence that a predictive model applied to an audio file obtained at the second listening site is appropriately applying weights to features of the audio file as compared to an audio file obtained at the first listening site in order to determine important relationships therebetween that are explained by anatomical changes. That is, without confirmation that the second listening site is within a reasonable threshold of the first listening site, a predictive model configured to provide insights with respect to the physical condition of the patient may inappropriately assign weight to a change in the sounds that may be attributable to a change in location as opposed to a change in physical condition.

In some embodiments, once the subsequent recording is confirmed to have been obtained at the target listening location, the subsequent recording is processed (e.g., one or more feature vectors are extracted from the subsequent recording file) and one or more predictive models are applied to the processed recording feature vectors. The one or more predictive models are trained and configured to identify possible changes in medical condition associated with the patient based on detected changes or features associated with lung or other body part sounds as part of the original and subsequent recordings. In some embodiments, the one or more predictive models may further take into account other anatomic parameters associated with the patient. Applying the one or more predictive models to the audio recordings provides for early detection and diagnoses associated with medical conditions that may otherwise have gone ignored because they are impossible to detect with the human ear or eye.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### VII. Examples

In some example embodiments, a system comprises memory and one or more processors communicatively coupled to the memory, the one or more processors configured to receive a first biometric recording data structure associated with a first patient listening site associated with a patient body. In some of these embodiments, the one or more processors are further configured to receive a second biometric recording data structure associated with a second patient listening site associated with the patient body. In some of these embodiments, the one or more processors are further configured to generate one or more first listening vectors based on the first biometric recording data structure and one or more second listening vectors based on the second biometric recording data structure. In some of these embodiments, the one or more processors are further configured to, responsive to determining, based on the one or more first listening vectors and the one or more second listening vectors, that the second patient listening site is different from the first patient listening site, initiate one or more guidance-related actions at a recording device to direct the recording device toward the first patient listening site for obtaining a subsequent biometric recording data structure.

In some of these embodiments, the first biometric recording data structure comprises one or more first audio recordings associated with a first constant sound source placed at a constant sound source site relative to the patient body.

In some of these embodiments, the second biometric recording data structure comprises one or more second audio recordings associated with a second constant sound source placed at the constant sound source site. In some of these embodiments, the one or more first audio recordings are associated with one or more organs or anatomic components of the patient body. In some of these embodiments, the one or more second audio recordings are associated with one or more organs or anatomic components of the patient body.

In some of these embodiments, the first listening vector comprises one or more distances between the first listening site and the first constant sound source and between the first listening site and one or more organs or anatomic components of the patient body.

In some of these embodiments, the second listening vector comprises one or more distances between the second listening site and the second constant sound source and between the second listening site and one or more organs or anatomic components of the patient body.

In some of these embodiments, the recording device comprises one or more transducers.

In some of these embodiments, the first constant sound source comprises one or more sound emitters.

In some of these embodiments, the second constant sound source comprises one or more sound emitters.

In some of these embodiments, the constant sound source site is a sternal notch of the patient body.

In some of these embodiments, the one or more processors are further configured to, responsive to determining, based on the first listening vector and the second listening vector, that the second patient listening site is equivalent to the first patient listening site, cause the recording device to provide audio, visual, or tactile feedback. In some of these embodiments, the tactile feedback comprises one or more of haptics or vibrations.

In some of these embodiments, the one or more guidance-related actions comprise one or more of haptics, vibrations, audio notification, or visual notification.

In some of these embodiments, the recording device is one or more of a stethoscope, a mobile computing device, a remote monitoring device, one or more audio capture devices, one or more video capture devices, or a computing device.

In some of these embodiments, the one or more processors are further configured to extract one or more feature vectors from the second biometric recording data structures, and apply one or more predictive models to the one or more feature vectors. In some of these embodiments, the one or more predictive models are configured to identify one or more changes in anatomic conditions associated with the patient body. In some of these embodiments, the one or more predictive models are trained based on historical anatomic data associated with a plurality of patient bodies.

In some example embodiments, one or more non-transitory computer-readable storage media include instructions that, when executed by one or more processors, cause the one or more processors to receive a first biometric recording data structure associated with a first patient listening site, receive a second biometric recording data structure associated with a second patient listening site, generate one or more first listening vectors based on the first biometric recording data structure and one or more second listening vectors based on the second biometric recording data structure, and responsive to determining, based on the one or more first listening vectors and the one or more second listening vectors, that the second patient listening site is different from the first patient listening site, initiate one or more guidance-related actions at a recording device to direct the recording device toward the first patient listening site.

In some example embodiments, a computer-implemented method comprises receiving, by one or more processors, a first biometric recording data structure associated with a first patient listening site, receiving, by the one or more processors, a second biometric recording data structure associated with a second patient listening site, generating, by the one or more processors, one or more first listening vectors based on the first biometric recording data structure and one or more second listening vectors based on the second biometric recording data structure, and responsive to determining, based on the one or more first listening vectors and the one or more second listening vectors, that the second patient listening site is different from the first patient listening site, initiating, by the one or more processors, one or more guidance-related actions at a recording device to direct the recording device toward the first patient listening site.

1. A system comprising memory and one or more processors communicatively coupled to the memory, the one or more processors configured to:
receive a first biometric recording data structure associated with a first patient listening site associated with a patient body;
receive a second biometric recording data structure associated with a second patient listening site associated with the patient body;
generate one or more first listening vectors based on the first biometric recording data structure and one or more second listening vectors based on the second biometric recording data structure; and
responsive to determining, based on the one or more first listening vectors and the one or more second listening vectors, that the second patient listening site is different from the first patient listening site, initiate one or more guidance-related actions at a recording device to direct the recording device toward the first patient listening site for obtaining a subsequent biometric recording data structure.

2. The system of claim 1, wherein the first biometric recording data structure comprises one or more first audio recordings associated with a first constant sound source placed at a constant sound source site relative to the patient body.

3. The system of claim 1, wherein the second biometric recording data structure comprises one or more second audio recordings associated with a second constant sound source placed at the constant sound source site.

4. The system of claim 2, wherein the one or more first audio recordings are associated with one or more organs or anatomic components of the patient body.

5. The system of claim 3, wherein the one or more second audio recordings are associated with one or more organs or anatomic components of the patient body.

6. The system of claim 2, wherein the first listening vector comprises one or more distances between the first listening site and the first constant sound source and between the first listening site and one or more organs or anatomic components of the patient body.

7. The system of claim 3, wherein the second listening vector comprises one or more distances between the second listening site and the second constant sound source and between the second listening site and one or more organs or anatomic components of the patient body.

8. The system of claim 1, wherein the recording device comprises one or more transducers.

9. The system of claim 2, wherein the first constant sound source comprises one or more sound emitters.

10. The system of claim 3, wherein the second constant sound source comprises one or more sound emitters.

11. The system of claim 2, wherein the constant sound source site is a sternal notch of the patient body.

12. The system of claim 1, wherein the one or more processors are configured to:
responsive to determining, based on the first listening vector and the second listening vector, that the second patient listening site is equivalent to the first patient listening site, cause the recording device to provide audio, visual, or tactile feedback.

13. The system of claim 12, wherein the tactile feedback comprises one or more of haptics or vibrations.

14. The system of claim 1, wherein the one or more guidance-related actions comprise one or more of haptics, vibrations, audio notification, or visual notification.

15. The system of claim 1, wherein the recording device is one or more of a stethoscope, a mobile computing device, a remote monitoring device, one or more audio capture devices, one or more video capture devices, or a computing device.

16. The system of claim 1, wherein the one or more processors are configured to:
extract one or more feature vectors from the second biometric recording data structures; and
apply one or more predictive models to the one or more feature vectors.

17. The system of claim 16, wherein the one or more predictive models are configured to identify one or more changes in anatomic conditions associated with the patient body.

18. The system of claim 17, wherein the one or more predictive models are trained based on historical anatomic data associated with a plurality of patient bodies.

19. One or more non-transitory computer-readable storage media including instructions that, when executed by one or more processors, cause the one or more processors to:
receive a first biometric recording data structure associated with a first patient listening site;
receive a second biometric recording data structure associated with a second patient listening site;
generate one or more first listening vectors based on the first biometric recording data structure and one or more second listening vectors based on the second biometric recording data structure; and
responsive to determining, based on the one or more first listening vectors and the one or more second listening vectors, that the second patient listening site is different from the first patient listening site, initiate one or more guidance-related actions at a recording device to direct the recording device toward the first patient listening site.

20. A computer-implemented method comprising:
receiving, by one or more processors, a first biometric recording data structure associated with a first patient listening site;
receiving, by the one or more processors, a second biometric recording data structure associated with a second patient listening site;
generating, by the one or more processors, one or more first listening vectors based on the first biometric recording data structure and one or more second listening vectors based on the second biometric recording data structure; and
responsive to determining, based on the one or more first listening vectors and the one or more second listening vectors, that the second patient listening site is different from the first patient listening site, initiating, by the one or more processors, one or more guidance-related actions at a recording device to direct the recording device toward the first patient listening site.
